SEQUENCE LISTING (108 sequences)

SEQ ID NO: 1 - DNA, 20 bp, Artificial Sequence
  Synthetic Primer
tctggagatcaggtggaagg

SEQ ID NO: 2 - DNA, 18 bp, Artificial Sequence
  Synthetic Primer
tgaacagctcgctgagat

SEQ ID NO: 3 - DNA, 20 bp, Artificial Sequence
  Synthetic Primer
cctcgctcttccgtgtctac

SEQ ID NO: 4 - DNA, 20 bp, Artificial Sequence
  Synthetic Primer
aacactaaatcccggcacac

SEQ ID NO: 5 - DNA, 20 bp, Artificial Sequence
  Synthetic Primer
tcagggtcctgaggtcaact

SEQ ID NO: 6 - DNA, 20 bp, Artificial Sequence
  Synthetic Primer
atagggagctgagggaatgc

SEQ ID NO: 7 - DNA, 20 bp, Artificial Sequence
  Synthetic Primer
ctcatcttcccgatggatgt

SEQ ID NO: 8 - DNA, 20 bp, Artificial Sequence
  Synthetic Primer
gccctggagcactatgaatc

SEQ ID NO: 9 - DNA, 20 bp, Artificial Sequence
  Synthetic Primer
tgaaggccaacctcctagtg

SEQ ID NO: 10 - DNA, 20 bp, Artificial Sequence
  Synthetic Primer
ccaagaagccatggaaagtc

SEQ ID NO: 11 - DNA, 20 bp, Artificial Sequence
  Synthetic Primer
aagtcacgtttgggaaatgg

SEQ ID NO: 12 - DNA, 20 bp, Artificial Sequence
  Synthetic Primer
gacatcaccagcagcctctc

SEQ ID NO: 13 - DNA, 20 bp, Artificial Sequence
  Synthetic Primer
gtccacctgcatcaggctat

SEQ ID NO: 14 - DNA, 20 bp, Artificial Sequence
  Synthetic Primer
gaagtcagagggacccacaa

SEQ ID NO: 15 - DNA, 20 bp, Artificial Sequence
  Synthetic Primer
tgcagattcgttttccctgt

SEQ ID NO: 16 - DNA, 20 bp, Artificial Sequence
  Synthetic Primer
cagagggacccacaaatcag

SEQ ID NO: 17 - DNA, 21 bp, Artificial Sequence
  Synthetic Primer
aaaaggaatgccagtggtgag

SEQ ID NO: 18 - DNA, 20 bp, Artificial Sequence
  Synthetic Primer
ccaaacatggttgcaggtta

SEQ ID NO: 19 - DNA, 20 bp, Artificial Sequence
  Synthetic Primer
agactgggttcgtgaattgg

SEQ ID NO: 20 - DNA, 21 bp, Artificial Sequence
  Synthetic Primer
tggttgcaggttattgacaca

SEQ ID NO: 21 - DNA, 23 bp, Artificial Sequence
  Synthetic Primer
agggaacatggaccactctgctg

SEQ ID NO: 22 - DNA, 25 bp, Artificial Sequence
  Synthetic Primer
tgggcatagaggacttccaatgtca

SEQ ID NO: 23 - DNA, 20 bp, Artificial Sequence
  Synthetic Primer
accactctgctgctcacctt

SEQ ID NO: 24 - DNA, 21 bp, Artificial Sequence
  Synthetic Primer
gctgggctttatcctgcacat

SEQ ID NO: 25 - DNA, 20 bp, Artificial Sequence
  Synthetic Primer
ttgggggcttctcttcatta

SEQ ID NO: 26 - DNA, 20 bp, Artificial Sequence
  Synthetic Primer
gaaagcccaaggtgtgaaga

SEQ ID NO: 27 - DNA, 20 bp, Artificial Sequence
  Synthetic Primer
gatggtgcctctctgctctc

SEQ ID NO: 28 - DNA, 20 bp, Artificial Sequence
  Synthetic Primer
gtgaagtccagccctgagtc

SEQ ID NO: 29 - DNA, 20 bp, Artificial Sequence
  Synthetic Primer
aggtgggctttcttctccat

SEQ ID NO: 30 - DNA, 20 bp, Artificial Sequence
  Synthetic Primer
agaggtggccctcttgttct

SEQ ID NO: 31 - DNA, 20 bp, Artificial Sequence
  Synthetic Primer
gttcctgataatggcgtgct

SEQ ID NO: 32 - DNA, 19 bp, Artificial Sequence
  Synthetic Primer
ggtccggaagtgacaagag

SEQ ID NO: 33 - DNA, 20 bp, Artificial Sequence
  Synthetic Primer
tcttggtgagacaggtggtg

SEQ ID NO: 34 - DNA, 20 bp, Artificial Sequence
  Synthetic Primer
gagaagggtattgggggaga

SEQ ID NO: 35 - DNA, 19 bp, Artificial Sequence
  Synthetic Primer
aaggggaccttcagcaatc

SEQ ID NO: 36 - DNA, 20 bp, Artificial Sequence
  Synthetic Primer
attgggggagatgcatagag

SEQ ID NO: 37 - DNA, 20 bp, Artificial Sequence
  Synthetic Primer
gggattagcgagcctttttc

SEQ ID NO: 38 - DNA, 20 bp, Artificial Sequence
  Synthetic Primer
acagctccccaccctaaaga

SEQ ID NO: 39 - DNA, 20 bp, Artificial Sequence
  Synthetic Primer
ttgctcctcacacacattcc

SEQ ID NO: 40 - DNA, 20 bp, Artificial Sequence
  Synthetic Primer
cagcaccaatctttctctgc

SEQ ID NO: 41 - DNA, 20 bp, Artificial Sequence
  Synthetic Primer
catcttggatggagggtttg

SEQ ID NO: 42 - DNA, 20 bp, Artificial Sequence
  Synthetic Primer
atctccccatctccaacctt

SEQ ID NO: 43 - DNA, 20 bp, Artificial Sequence
  Synthetic Primer
gactgcctctcctcttgtgc

SEQ ID NO: 44 - DNA, 20 bp, Artificial Sequence
  Synthetic Primer
atgcacccataggcaagact

SEQ ID NO: 45 - DNA, 20 bp, Artificial Sequence
  Synthetic Primer
gaagtgggggagaagaatcc

SEQ ID NO: 46 - DNA, 20 bp, Artificial Sequence
  Synthetic Primer
aacttccaggaggagggtgt

SEQ ID NO: 47 - DNA, 20 bp, Artificial Sequence
  Synthetic Primer
agaaccctcagaccacgatg

SEQ ID NO: 48 - DNA, 20 bp, Artificial Sequence
  Synthetic Primer
aggagggtgttgagtgttgg

SEQ ID NO: 49 - DNA, 20 bp, Artificial Sequence
  Synthetic Primer
ttctgtctgctgcaaagtgg

SEQ ID NO: 50 - DNA, 20 bp, Artificial Sequence
  Synthetic Primer
tcatgaggctctgacattgc

SEQ ID NO: 51 - DNA, 20 bp, Artificial Sequence
  Synthetic Primer
gccacaaacctaacgtgctt

SEQ ID NO: 52 - DNA, 20 bp, Artificial Sequence
  Synthetic Primer
atctggcagcacacaccata

SEQ ID NO: 53 - DNA, 20 bp, Artificial Sequence
  Synthetic Primer
gaagcacagctcggtttctc

SEQ ID NO: 54 - DNA, 20 bp, Artificial Sequence
  Synthetic Primer
agctccaggtcagcaagatg

SEQ ID NO: 55 - DNA, 20 bp, Artificial Sequence
  Synthetic Primer
tctgaatgtctcccctggtc

SEQ ID NO: 56 - DNA, 20 bp, Artificial Sequence
  Synthetic Primer
atcccaggtgccagtgacta

SEQ ID NO: 57 - DNA, 20 bp, Artificial Sequence
  Synthetic Primer
gccagcatggctaattgaac

SEQ ID NO: 58 - DNA, 20 bp, Artificial Sequence
  Synthetic Primer
ggactaagcagggagggagt

SEQ ID NO: 59 - DNA, 20 bp, Artificial Sequence
  Synthetic Primer
tcacgcaggttccctatctc

SEQ ID NO: 60 - DNA, 20 bp, Artificial Sequence
  Synthetic Primer
ggagggagtgacaccttgaa

SEQ ID NO: 61 - DNA, 20 bp, Artificial Sequence
  Synthetic Primer
cccagtgaccccctaacttt

SEQ ID NO: 62 - DNA, 20 bp, Artificial Sequence
  Synthetic Primer
ttagcttggtgggaggactg

SEQ ID NO: 63 - DNA, 20 bp, Artificial Sequence
  Synthetic Primer
cccccatgttaaactctgct

SEQ ID NO: 64 - DNA, 20 bp, Artificial Sequence
  Synthetic Primer
gggaggactgacctaagcaa

SEQ ID NO: 65 - DNA, 20 bp, Artificial Sequence
  Synthetic Primer
tccagtggctatgggaccta

SEQ ID NO: 66 - DNA, 20 bp, Artificial Sequence
  Synthetic Primer
catgacccacctttcacaca

SEQ ID NO: 67 - DNA, 20 bp, Artificial Sequence
  Synthetic Primer
ctaggaacagggaccaccag

SEQ ID NO: 68 - DNA, 20 bp, Artificial Sequence
  Synthetic Primer
aaaaccgaatccagggtgtt

SEQ ID NO: 69 - DNA, 20 bp, Artificial Sequence
  Synthetic Primer
ctcatggtcccctgaaaaga

SEQ ID NO: 70 - DNA, 20 bp, Artificial Sequence
  Synthetic Primer
tcaccatcgtgatggacact

SEQ ID NO: 71 - DNA, 20 bp, Artificial Sequence
  Synthetic Primer
aaggagacctggtgtggttg

SEQ ID NO: 72 - DNA, 20 bp, Artificial Sequence
  Synthetic Primer
tcgtgatggacactgaagga

SEQ ID NO: 73 - DNA, 20 bp, Artificial Sequence
  Synthetic Primer
tcagagctcaggggaggata

SEQ ID NO: 74 - DNA, 20 bp, Artificial Sequence
  Synthetic Primer
gagtctgcggtgctgtgata

SEQ ID NO: 75 - DNA, 20 bp, Artificial Sequence
  Synthetic Primer
ggaagtggcctgtgtagtgc

SEQ ID NO: 76 - DNA, 20 bp, Artificial Sequence
  Synthetic Primer
acattcagcccctacactgc

SEQ ID NO: 77 - DNA, 20 bp, Artificial Sequence
  Synthetic Primer
cccagctgcctcattattgt

SEQ ID NO: 78 - DNA, 17 bp, Artificial Sequence
  Synthetic Primer
accatcaagggtgtcca

SEQ ID NO: 79 - DNA, 20 bp, Artificial Sequence
  Synthetic Primer
gcctcattattgtggcctgt

SEQ ID NO: 80 - DNA, 20 bp, Artificial Sequence
  Synthetic Primer
ggacaacacgatttcccttg

SEQ ID NO: 81 - DNA, 20 bp, Artificial Sequence
  Synthetic Primer
acctgctcaccagcaagatt

SEQ ID NO: 82 - DNA, 20 bp, Artificial Sequence
  Synthetic Primer
tccattctcttccagccagt

SEQ ID NO: 83 - DNA, 20 bp, Artificial Sequence
  Synthetic Primer
ctgctgcccatgtttacaga

SEQ ID NO: 84 - DNA, 20 bp, Artificial Sequence
  Synthetic Primer
cttccatccttgctcctgtc

SEQ ID NO: 85 - DNA, 22 bp, Artificial Sequence
  Synthetic Primer
catttcccctaatccttttcca

SEQ ID NO: 86 - DNA, 22 bp, Artificial Sequence
  Synthetic Primer
gtgatcccagatttaggccttc

SEQ ID NO: 87 - DNA, 22 bp, Artificial Sequence
  Synthetic Primer
ggaaatatagggaagggaaggn

SEQ ID NO: 88 - DNA, 22 bp, Artificial Sequence
  Synthetic Primer
ttgacagggtaccaggagatga

SEQ ID NO: 89 - DNA, 22 bp, Artificial Sequence
  Synthetic Primer
gcctctcgtggtttgttttgtc

SEQ ID NO: 90 - DNA, 22 bp, Artificial Sequence
  Synthetic Primer
cccagggtagggtccaataatc

SEQ ID NO: 91 - DNA, 20 bp, Artificial Sequence
  Synthetic Primer
ctgaaccgccaaggactcat

SEQ ID NO: 92 - DNA, 23 bp, Artificial Sequence
  Synthetic Primer
ttttccctccctactaacacacg

SEQ ID NO: 93 - DNA, 19 bp, Artificial Sequence
  Synthetic Primer
cctgctctcctcctgaacc

SEQ ID NO: 94 - DNA, 20 bp, Artificial Sequence
  Synthetic Primer
cagggatacctggaggatga

SEQ ID NO: 95 - DNA, 20 bp, Artificial Sequence
  Synthetic Primer
agcagggcagatgcttaatg

SEQ ID NO: 96 - DNA, 20 bp, Artificial Sequence
  Synthetic Primer
ctggaggatgatggctgact

SEQ ID NO: 97 - DNA, 18 bp, Artificial Sequence
  Synthetic Primer
gaatggggtggggtgtct

SEQ ID NO: 98 - DNA, 20 bp, Artificial Sequence
  Synthetic Primer
cagtcacattcgcatcttgg

SEQ ID NO: 99 - DNA, 20 bp, Artificial Sequence
  Synthetic Primer
ggtcctttggagtgctgcta

SEQ ID NO: 100 - DNA, 20 bp, Artificial Sequence
  Synthetic Primer
ccatctgccccttcatctta

SEQ ID NO: 101 - DNA, 20 bp, Artificial Sequence
  Synthetic Primer
cctttacacctgcgcactct

SEQ ID NO: 102 - DNA, 20 bp, Artificial Sequence
  Synthetic Primer
aaatgggcaaatggagacac

SEQ ID NO: 103 - DNA, 20 bp, Artificial Sequence
  Synthetic Primer
gttcgcaccctcaacgtatt

SEQ ID NO: 104 - DNA, 20 bp, Artificial Sequence
  Synthetic Primer
gcaaatggagacaccagtca

SEQ ID NO: 105 - DNA, 21 bp, Artificial Sequence
  Synthetic Primer
aaatcctggtttcctcatctg

SEQ ID NO: 106 - DNA, 20 bp, Artificial Sequence
  Synthetic Primer
gtgtggctccttctttgcta

SEQ ID NO: 107 - DNA, 20 bp, Artificial Sequence
  Synthetic Primer
aagggggacacgtgaatatg

SEQ ID NO: 108 - DNA, 19 bp, Artificial Sequence
  Synthetic Primer
tggcacaaaacaaaacgtg

(19) United States
(12) Patent Application Publication
(10) Pub. No.: US 2012/0129869 A1
(43) Pub. Date: May 24, 2012

(54) PHOSPHORYLATION AND MUTATIONS OF ANAPLASTIC LYMPHOMA KINASE AS A DIAGNOSTIC AND THERAPEUTIC TARGET IN LUNG CANCER

(75) Inventors: Yuh-Shan Jou, Taipei (TW); Yi-Wei Wang, Taipei (TW)

(73) Assignee: Academia Sinica, Taipei (TW)

(21) Appl. No.: 13/294,588

(22) Filed: Nov. 11, 2011

Related U.S. Application Data
(60) Provisional application No. 61/412,858, filed on Nov. 12, 2010.

(51) Int. Cl. A61K 31/506; G01N 33/483; A61P 35/00; A61K 31/517; A61K 31/4545; G01N 33/574; C12Q 1/02

(52) U.S. Cl. 514/252.18; 435/7.1; 436/501; 800/3; 435/29; 514/266.4; 514/318

(57) ABSTRACT

The invention related to the use of high-density loss of heterozygosity (LOH) mapping in lung adenocarcinoma to identify intragenic LOH and driver mutations in different domains of ALK resulted in enhanced tumor growth in xenografted mouse. Mutant (H694R and E1384K) ALKs showed activation of Y1604 ALK and downstream AKT, STAT3 and ERK signaling pathways. Increases of oncogenic signalings resulted in enhanced cell proliferation, colony-formation, cell-migration and tumor-growth in xenografted mouse. Western blot and immunohistochemistry analysis using antibody against phospho-Y1604 ALK on 11 lung cancer cell-lines and 263 cancer specimens indicated ALK activation in all lung cancers regardless of tumor stages. Treating mutant-bearing mice with ALK inhibitor WHI-P 154 resulted in tumor shrinkage, metastasis suppression, and improved survival. Hyperphosphorylation of Y1604 ALK occurred early and continuously throughout tumor progression and could be used as a biomarker to detect lung cancer. Oncogenic ALK point mutations could be treatment targets for lung cancer.

### CROSS-REFERENCE TO RELATED APPLICATION

This is a non-provisional application of U.S. Provisional Application Ser. No. 61/412,858, filed Nov. 12, 2010.

### Abbreviations

Ad: adenocarcinoma; AIG: anchorage-independent growth; Akt: v-akt murine thymoma viral oncogene homolog 1/PKB; ALCL: anaplastic large cell lymphoma; ALK: anaplastic lymphoma kinase; BRAF: V-raf murine sarcoma viral oncogene homolog B1; CLTC-ALK: clathrin heavy polypeptide-anaplastic lymphoma kinase; c-Met: mesenchymal-epithelial transition factor; EGFR: epidermal growth factor receptor; EML4: echinoderm microtubule-associated protein-like 4; EML4-ALK: echinoderm microtubule-associated protein-like 4-anaplastic lymphoma kinase; ERK: Extracellular Signal-Regulated Kinase; HER2: V-erb-b2 erythroblastic leukemia viral oncogene homolog 2/ErbB-2; IHC: immunohistochemistry; JAK3: Janus kinase 3; K-ras: Kirsten rat sarcoma viral oncogene homolog; LKB1: STK11/serine threonine-protein kinase 11; NPM: nucleophosmin; NPM-ALK: nucleophosmin-anaplastic lymphoma kinase; NSCLC: non-small lung cancer; PI3K: Phosphatidylinositol-3-Kinase; PIK3CA: Phosphoinositide-3-kinase catalytic subunit; SCLC: small cell lung cancer; SQ: squamous carcinoma; STAT3: Signal Transducer and Activator of Transcription 3; TPM4-ALK: tropomyosin 4-anaplastic lymphoma kinase.

### TECHNICAL FIELD

This invention relates to diagnosis of lung cancer identifying novel ALK mutations from lung adenocarcinoma patients and detecting various tumorigenic activities through a panel of assays. The invention also relates to treatment of lung cancer using inhibitors to suppress ALK mutation-mediated tumorigenesis.

### BACKGROUND OF THE INVENTION

Lung cancer, the leading cause of cancer mortality worldwide resulted in 1.3 million deaths annually, can be broadly classified to non-small cell lung cancers (NSCLC) and small cell lung cancers (SCLC) which accounts for 85% and 15% cases, respectively (WHO). Among NSCLC, the number of patients developing adenocarcinoma constituted for more than 40% of lung cancer patients is increasing in recent decades and replaces squamous cell carcinoma to become the major subtype of lung cancer. Recent advances of molecular genetic studies in lung cancer revealed many genes with somatic alterations including p53, K-ras, EGFR, HER2, c-MET, LKB1, PIK3CA, and BRAF that triggered selective advantages of cancer cells to promote tumor growth, apoptotic resistance, angiogenesis and metastasis. Only EGFR mutations become a promising target for lung cancer therapy. EGFR mutations are important predicative factors for successful response to small molecule EGFR tyrosine kinase inhibitors such as gefitinib and erlotinib. However, its prognostic impacts in lung cancer remain controversial. The overall lung cancer 5-year survival rate remains around 15%. Therefore, discovery of novel therapeutic targets for lung cancer are urgently needed. In our study, we identified 6 novel ALK mutations from lung adenocarcinoma patients and detected significantly enhanced tumorigenic activities through a panel of assays. Two oncogenic mutants H694R and E1384K were selected to demonstrate the strong activation of downstream ALK tumorigenic signalings. More importantly, the mutations-mediated tumorigenesis can be suppressed by ALK inhibitor suggesting that lung cancer patients harboring ALK mutations can be treated with ALK inhibitor for a new therapeutic strategy to improve patient survival.

### SUMMARY OF THE INVENTION

Because lung cancer is the leading cause of cancer mortality worldwide, it is important to discover a new biomarker for early detection of lung cancer. We demonstrated that up-regulated expression of phosphorylated-Y1604 ALK is a common phenomenon in lung cancer regardless of stages and different subtypes. High specificity and sensitivity of detecting phosphorylated-Y1604 ALK in lung cancer could serve as an early diagnostic marker for lung cancer. The invention also provides treatment of lung cancer using inhibitors targeting ALK mutation-mediated tumorigenesis.

Unique Features of the Invention/Advantages when Compared to the Existing Technologies

Even with the high response rate of small molecule EGFR tyrosine kinase inhibitors such as gefitinib and erlotinib to lung cancer patients with EGFR mutations, the prognosis of lung cancer patients remained unchanged with an overall 5-year survival rate around 15%. With potent oncogenic activity of ALK mutations, our experimental results demonstrated that ALK could be a promising target for monotherapy or combination therapy with other mutated targets such as EGFR to treat the most fatal and common human cancer.

In order to improve the poor survival rate of lung cancer, it's important to develop a new diagnostic marker for early detection of lung cancer. According to our experimental results, we suggest that increase expression of phosphorylated-Y1604-ALK is a good candidate for detecting the occurrence of lung cancer. The reason is that in normal lung tissues the average phosphorylated-Y1604-ALK scoring intensity is 0.554±0.3340; in contrast, in lung cancer the average scoring is dramatically increased to 2.9684±0.6852. The sensitivity and specificity of phoshorylated-Y1604-ALK in lung cancer was also calculated. The sensitivity and specificity of phosphorylated-Y1604-ALK antibody for detecting lung cancer with IHC intensity >2 are 92.8% and 100%, respectively. If we set the IHC intensity score >1, the sensitivity and specificity of phosphorylated-Y1604-ALK antibody for detecting lung cancer are 99.6% and 89.2%, respectively. More importantly, the IHC assay is a routine procedure in all hospitals, suggesting that the examination of phoshorylated-Y1604-ALK is a convenient and efficient way for detecting lung cancer in clinic.

### Commercial Applications of the Invention

(I). ALK mutations could serve as diagnostic and therapeutic target for clinical intervention in monotherapy or in combination with other therapeutic modalities. It can also serve as a useful target for developing new ALK inhibitors for treatment of lung cancer.

(II). The detection of up-regulated phosphorylated-Y1604-ALK expression level by IHC assay can be applied to detect early and all stages of lung cancer regardless of subtypes.

### DETAILED DESCRIPTION OF THE INVENTION AND EMBODIMENTS

### Introduction

Lung cancer, the leading cause of cancer mortality worldwide resulted in 1.3 million deaths annually, can be broadly classified to non-small cell lung cancers (NSCLC) and small cell lung cancers (SCLC) which accounts for 85% and 15% cases, respectively¹. Among NSCLC, the number of patients developing adenocarcinoma constituted for more than 40% of lung cancer patients is increasing in recent decades and replaces squamous cell carcinoma to become the major subtype of lung cancer². Recent advances of molecular genetic studies in lung adenocarcinoma revealed many genes with somatic alterations including p53, K-ras, EGFR, HER2, c-MET, LKB1, PIK3CA, and BRAF that triggered selective advantages of cancer cells to promote tumor growth, apoptotic resistance, angiogenesis and metastasis. Depending on tumor subtype, ethnicity, smoking status and gender, EGFR mutations (<40%) are commonly observed in Asian female non-smoking adenocarcinoma patients but low frequency in non-Asian patients. In contrast, K-ras (<30%) and LKB1 (<34%) mutations are frequently detected in non-Asian and smoking patients but low frequency in Asian patients³⁻⁴. EGFR mutations are important predicative factors for successful response to small molecule EGFR tyrosine kinase inhibitors such as gefitinib and erlotinib⁵⁻⁶. However, its prognostic impacts in lung adenocarcinoma remain controversial. Even with recent therapeutic advances, the overall 5-year survival rate for lung adenocarcinoma remains around 15%⁷. Therefore, discovery of novel targets for developing therapeutic strategies are urgently needed.

ALK was initially identified in a chromosomal translocation t(2; 5)(p23; q35) associated with approximately 75% anaplastic large cell lymphoma (ALCL) patients⁸⁻⁹. Translocation resulted in a chimeric tyrosine kinase NPM-ALK (5′ nucleophosmin fused to 3′ ALK) with constitutive oncogenic activity to enhance cell proliferation and migration, to resist apoptotic signaling, and to rearrange cytoskeleton for changing cell shape through activation of multiple interconnected intracellular signaling pathways including Ras/ERK, JAK3/STAT3, and PI3K/AKT pathways¹⁰. Recently, another fusion oncogene EML4-ALK (5′ echinoderm microtubule-associated protein-like 4 fused to 3′ ALK) was identified in lung adenocarcinoma with characteristics of ligand-independent and constitutive active tyrosine kinase which possesses oncogenic activity in vitro and in vivo¹¹. Inhibitors of ALK kinase were shown to suppress tumors in EML4-ALK transgenic and xenografted lung cancer models, which support EML4-ALK as a novel driver mutation and therapeutic target in NSCLC¹². Multiple EML4-ALK fusion variants have been described mainly in adenocarcinoma with prevalence up to 7% of total lung cancers¹³. Other ALK alterations also observed in inflammatory myofibroblastic tumors (TPM4-ALK), diffused large B-cell lymphoma (CLTC-ALK) and recently in sporadic and familial neuroblastoma (gain-of-function mutations)¹⁴⁻¹⁸. Since ALK is not widely expressed in adult tissues except a few neuronal cells and since several novel ALK-selective small molecule inhibitors are currently in preclinical or early clinical trials, targeting aberrant ALK to abrogate oncogenic pathways will have tremendous impacts for cancer therapy.

To directly illustrate functional alterations of ALK mutations in lung adenocarcinoma, we applied a panel of experiments to assess 6 non-synonymous ALK mutations using ALK kinase activation, cell proliferation, migration, and anchorage-independent growth assays. In combination with in vivo mouse xenograft tumorigenesis assay, we identified several oncogenic ALK mutations. Two strong transforming ALK mutations were selected for detail analysis of aberrant signal transductions and potential therapeutic interventions by treatment with ALK inhibitor. We provided lines of evidence that ALK kinase activation by phosphorylation is one of the most common events in lung cancer. Oncogenic ALK mutations could lead to malignant tumor formation, metastasis and poor survival in mouse xenograft and systemic metastasis models. Treatment of ALK inhibitor to the cells and mouse models not only suppress tumorigenesis and metastasis but also prolong survival of ALK mutant-bearing mice.

### Result

### Identification and Tumorigenicity of ALK Somatic Alterations

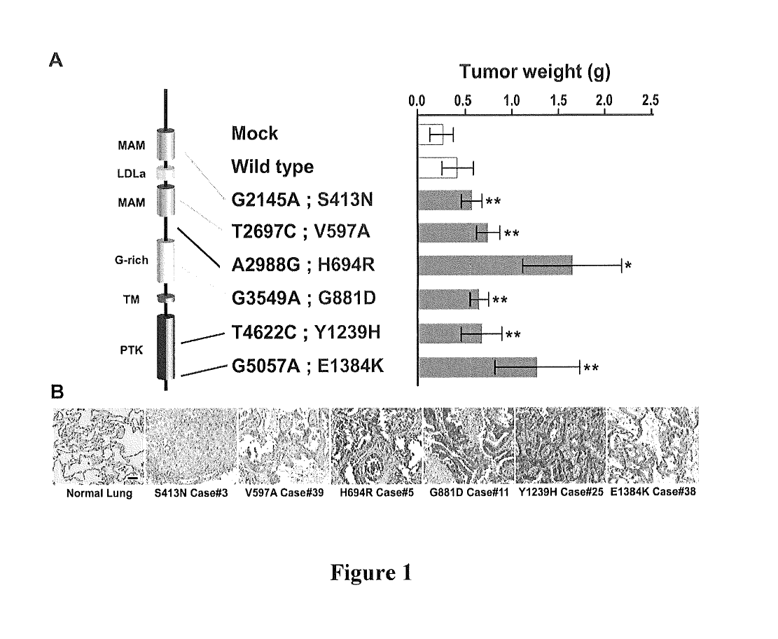

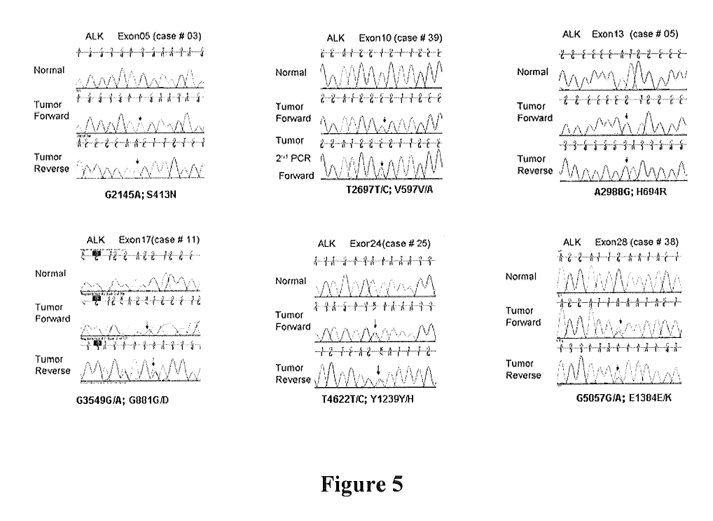

After increase of microsatellite marker density for refined mapping of loss of heterozygosity (LOH) regions, we found that microsatellite markers with LOH frequencies of 54.3% (AFM102ya1, 19/35), 69.4% (AFM220YH4, 25/36) and 69.4% (AFM198wc5, 25/36) were located within known lung cancer genes of EGFR, FHIT and ALK, respectively. Since 2p23 chromosomal region where ALK located was found to be amplified in comparative genome hybridization (CGH) analysis¹⁹⁻²¹, we suggested that ALK conferred unequal allelic amplification resulted in frequent LOH and therefore selected for detection of somatic point mutations. Although no ALK mutations were detected in 11 lung cancer cell lines, we found 6 ALK mutations located in different protein domains (S413N at MAM1 domain, V597A at MAM2, G881D at Glycine-rich domain, Y1239H and E1384K at kinase domain and H694R at non-domain area) and displayed tumorigenic effects in transfectants of H1299 xenograft tumor models (FIG. 1A). Comparing the tumor weights with the parental H1299 cell after 5 weeks injection, over-expressed wild type ALK moderately increased tumor weight but all 6 mutations showed significant increase of tumor size (FIG. 1A). All somatic ALK mutations were further confirmed by forward and reverse DNA sequencing (Supplementary FIG. 5) and validated by increased phospho-Y1604 ALK expression²² using polymer-amplified immunohistochemical (IHC) analysis on mutation-bearing tissue sections from six patients (FIG. 1B).

### E694R and E1384K Mutants Display Constitutive Kinase Activity

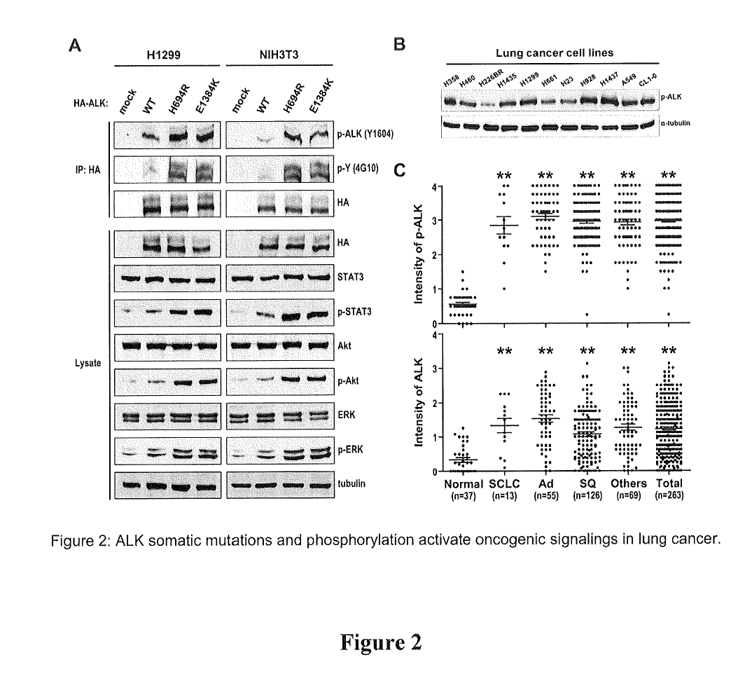

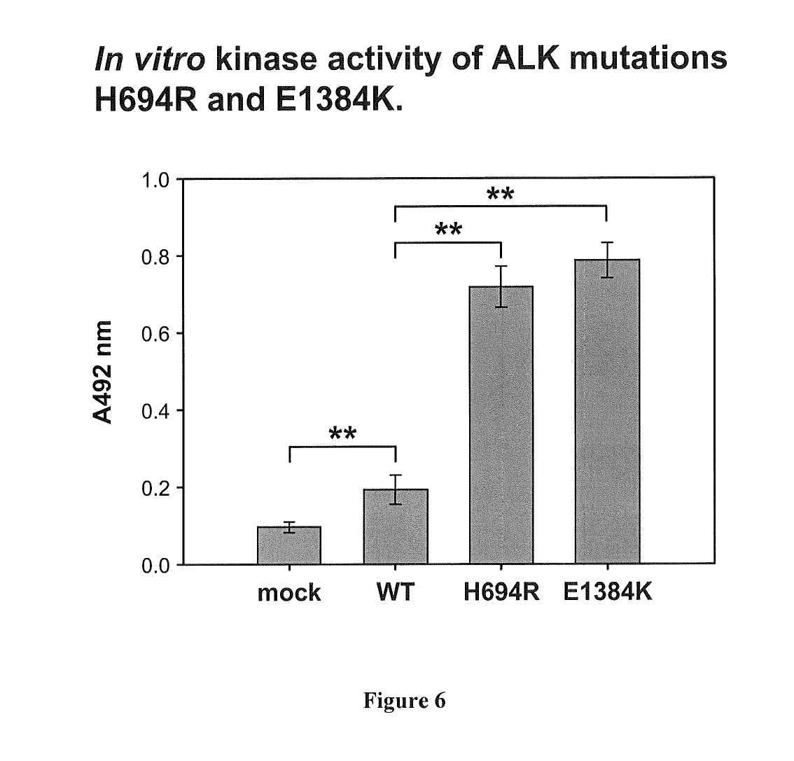

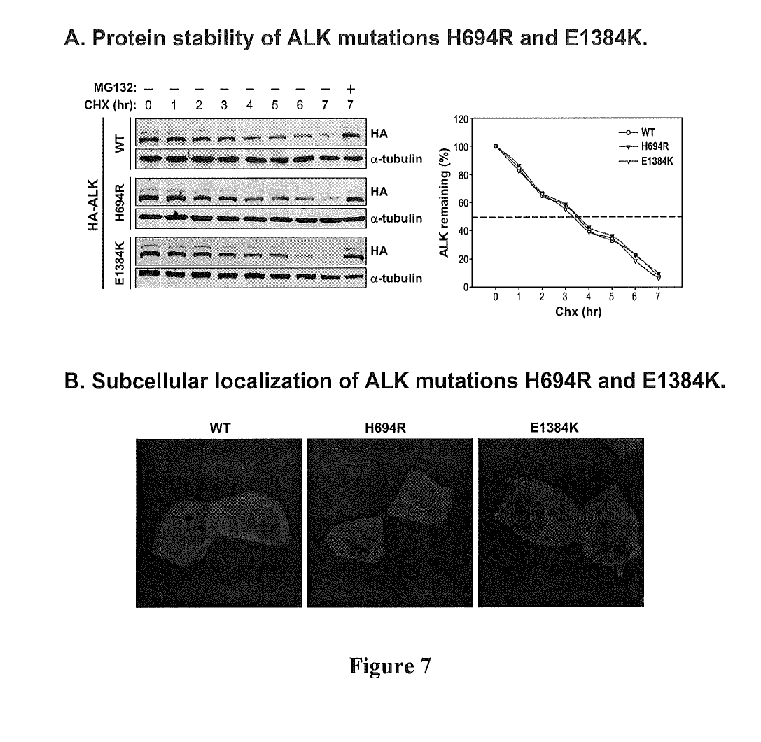

To further reveal the mutation-mediated alterations of ALK kinase activity and downstream cellular signaling, H694R at extracellular region and E1384K at kinase domain were stably expressed in both H1299 and the mouse embryonic NIH3T3 fibroblasts for demonstration of oncogenic effects of mutations per se without restriction in lung cancer genetic background. Using immunoprecipitation and Western blotting analysis, we showed that wild type ALK transfectants in H1299 and NIH3T3 cells slightly increase Y1604 and overall tyrosine phosphorylation but both H694R and E1384K transfectants significantly enhanced tyrosine phosphorylations (FIG. 2A). The enhanced tyrosine kinase activity was further validated by in vitro kinase assay (FIG. 6). To further elucidate the altered signaling pathways, we examined the activations of ALK downstream effectors including STAT3, Akt and ERK. In consistent with the increased kinase activity, H694R and E1384K showed strong protein phosphorylation of STAT3, Akt and ERK as compared with wild type and mock controls (FIG. 2A). To further realize the molecular mechanism of mutations-enhanced ALK kinase activity, we examined H694R and E1384K protein stability and subcellular localization. Our results showed that H694R and E1384K mutant and wild type ALK have similar protein properties with protein half-life about 3.5 hours and uniform cytoplasmic localization (FIG. 7). Taken together, our findings indicated that, without altering protein stability and subcellular localization, H694R and

E1384K mutants per se constitutively activate ALK kinase activity and downstream STAT3, Akt and ERK signaling pathways in both H1299 and NIH3T3 cells.

### Increased Expression of Phosphorylated-Y1604 ALK is a Diagnostic Marker for Lung Cancer

Given that ALK mutations showed increased expression of phosphor-Y1604 ALK and kinase activity in cell models and patient tissues, we investigated whether activation of endogenous ALK kinase activity existed in lung cancer cell lines and clinical specimens. Although we have successfully detected phosphor-Y1604 ALK and total ALK expression in tissue sections of patients using polymer-based signal enhancement technology in IHC assay, we failed to detect endogenous ALK in 11 lung cancer cell lines using antibodies from 3 different suppliers in Western blot analysis. In contrast, we demonstrated that hyperphosphorylation of Y1604 ALK was easily detected in 11 lung cancer cell lines (FIG. 2B).

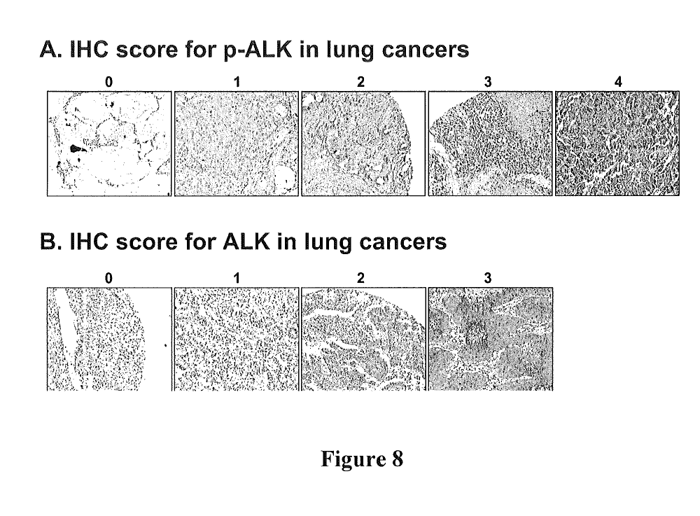

To further examine the expression of activated ALK kinase in clinical specimens of lung cancers, IHC staining of phosphorylated-Y1604 ALK was investigated using lung cancer tissue arrays containing 37 normal and 263 lung cancer tissues including 13 small cell lung cancers, 55 adenocarcinomas, 126 squamous carcinomas and 69 other subtypes of lung cancers. The staining intensity was evaluated by two pathologists and assigned a score according to staining intensity ranged from 0˜4 (FIG. 8). Regardless of lung cancer subtypes and stages, all tumor tissues showed significantly higher intensities of phosphorylated-Y1604 ALK expression than that in normal lung tissues with average scoring intensity of 2.9684±0.6852 versus 0.5541±0.3340, respectively. (p<0.001). The sensitivity of phosphorylated-Y1604 ALK antibody detected lung cancer specimens with IHC scoring intensity >1 and >2 are 99.6% and 92.8%, respectively. The same lung cancer specimens were also examined with total ALK expression by IHC analysis. Regardless of lung cancer subtypes and stages, we observed sensitivity only 61.59% (162/263) and 18.3% (48/263) to detect lung cancer with IHC scoring intensity >1 and >2, respectively. Statistic analysis of IHC results indicated that there is no correlation of expression between phosphorylated-Y1604 and total ALK in lung cancer samples (p=0.4449, Table 2). Our results demonstrated that activation of ALK kinase plays important roles not only in adenocarcinoma but also in other subtypes of lung cancers. The increased expression of phosphorylated-Y1604 ALK could be an early step in lung cancer development and potentially be a useful diagnostic marker for lung cancer (FIG. 2C).

### Tumorigenic Signaling of H694R and E1384K Mutations in Mouse Xenograft Models

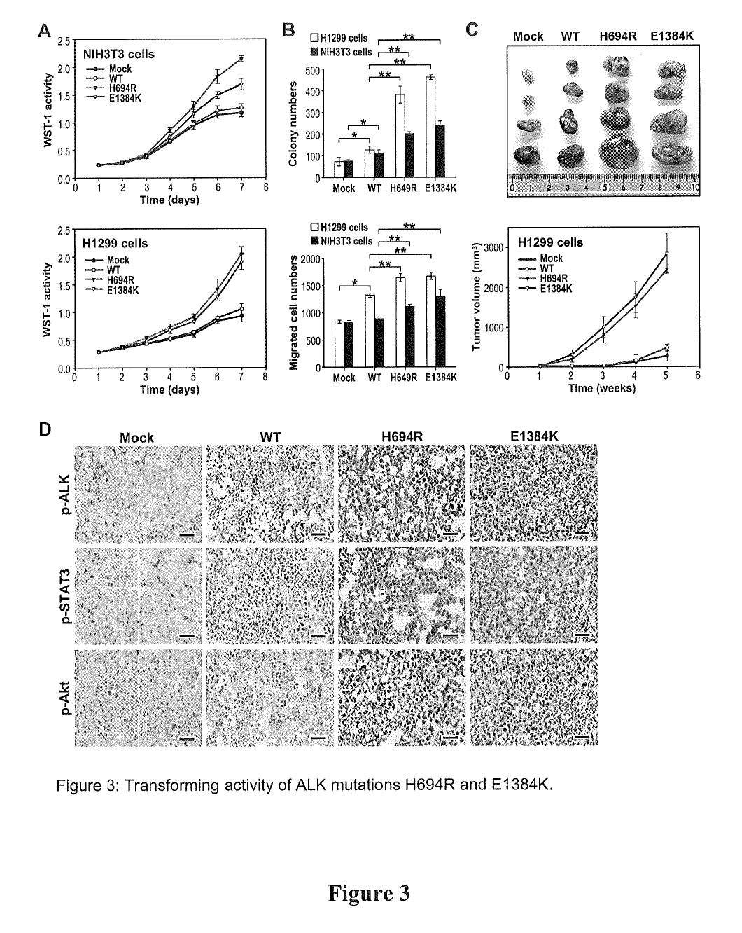

To demonstrate detail gains of oncogenic effects of ALK mutations, in vitro and in vivo assays of tumorigenicity were conducted in H694R and E1384K transfectants of H1299 and NIH3T3 cells. Our results showed that expression of wild type ALK only slightly enhanced tumorigenic effects in comparison with mock controls, especially in longer assayed time points (FIG. 3A-3C). In contrast, expression of H694R and E1384K mutations significantly increased tumorigenic effects as compared with that of wild type ALK (FIG. 3A-3C). In order to further demonstrate the tumorigenic induction of ALK mutations is due to their increased kinase activity, we performed IHC staining on tumor sections from xenograft mice models using antibodies against activated ALK kinase and its downstream signaling mediators STAT3 and Akt. Our results consistently showed that the ALK kinase activity as measured by the phosphorylated proteins of ALK, STAT3 and Akt only slightly increased in wild type but significant up-regulated in H694R and E1384K mutant-bearing xenografted tumor sections (FIG. 3D). Together, these results suggested that tumor progression driven by ALK mutations H694R and E1384K could be attributed to their constitutive kinase activities and increased downstream tumorigenic signalings.

### H694R and E1384K Mutants are Sensitive to ALK Inhibitor

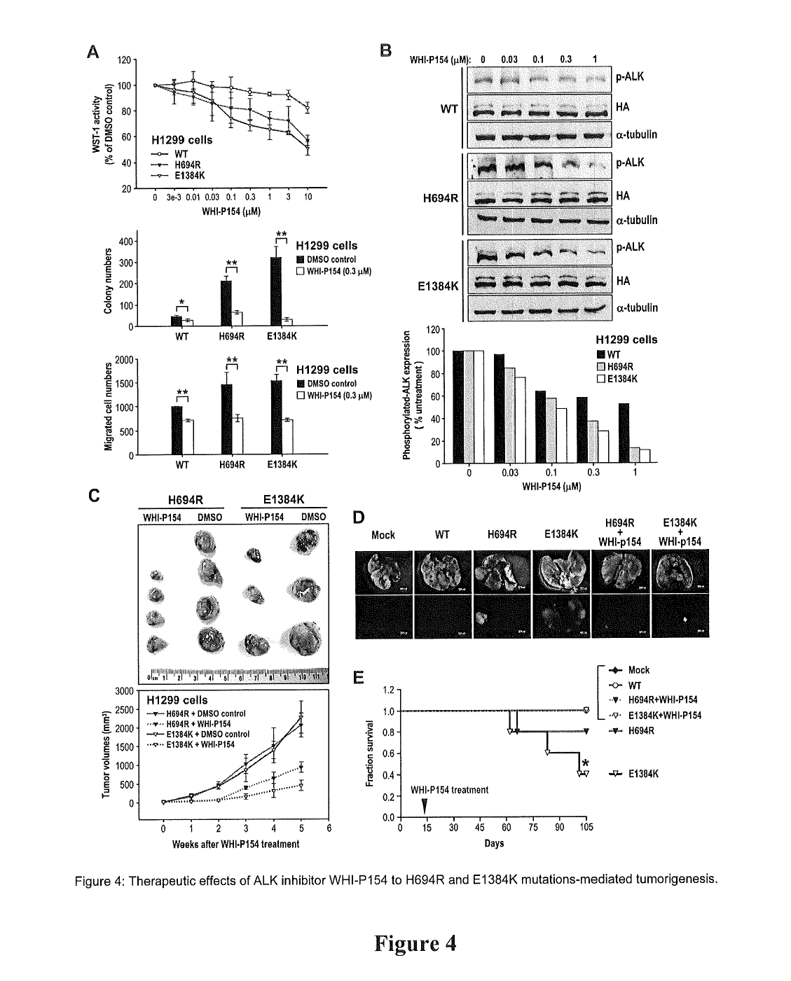

To investigate whether small molecule ALK kinase inhibitor can be applied to suppress ALK mutations-mediated tumorigenicity, we next investigated the sensitivity of wild type, H694R and E1384K ALK to the inhibitor WHI-P154 which recently was shown to repress ALK kinase activity²³. Our results demonstrated that WHI-P154 treatments showed a dose-dependent inhibition of cell growth and expression of phosphorylated-Y1604 ALK to both wild type and mutated ALK transfectants. However, mutations H694R or E1384K represented higher inhibitory sensitivity than wild type (FIGS. 4A and 4B). The inhibitory effects of WHI-P154 on ALK kinase activity were also tested using the AIG and cell migration assays in H1299 cells. Consistently, our results showed that WHI-P154 treatments resulted in profound inhibition of soft agar growth and cell migration as compared with DMSO control (FIG. 4A). The 2.28˜2.86 folds lower WHI-P154 half-maximal inhibitory concentration (IC₅₀) to H694R and E1384K mutants than wild type ALK indicated that both ALK mutants are sensitive to ALK inhibitor WHI-P154 with higher inhibitory response than wild type ALK. Our results suggested that oncogenic ALK mutations could be a potential therapeutic target in lung adenocarcinomas.

### WHI-P154 Inhibits H694R and E1384K Mutation-Mediated Tumorigenesis and Metastasis and Prolongs Survival of Mutation-Bearing Mice

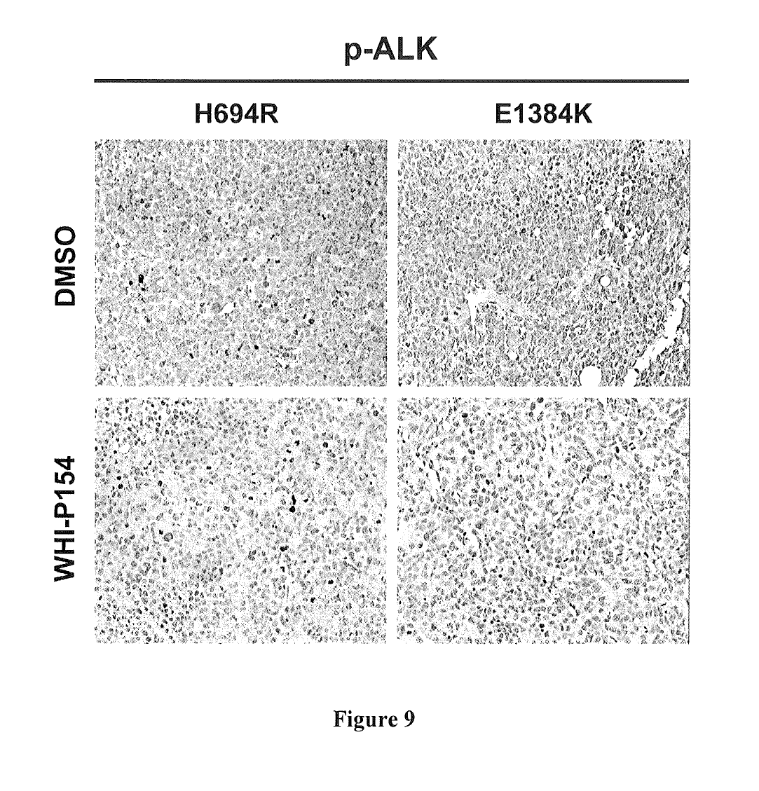

To further evaluate therapeutic potential of inhibiting constitutive kinase activity of ALK mutants, we next examined the in vivo therapeutic effect of WHI-P154 on H694R and E1384K in H1299 transfectants using subcutaneous injected nude mice xenograft model and tail vein injected lung metastasis model. In tumorigenesis model, once the xenografted tumors developed to tumor volumes around 30˜50 mm³, mice were randomly divided into two groups and treated with WHI-P154 or DMSO control daily. WHI-P154-treated ALK mutations-bearing mice showed significant reduction of tumor growth as compared with control mice (FIG. 4C). In order to further confirm that the WHI-P154 inhibition of xenografted tumor growth is due to decrease of ALK kinase activity, tumor sections from xenografted mice were examined by IHC staining against phosphorylated-Y1604 ALK. In consistent with the kinase-dependent oncogenic activity of ALK mutations, significant lower expression of phosphorylated-Y1604 ALK was detected in WHI-P154-treated tumor sections of both H694R and E1384K mutations as compared with DMSO control (FIG. 9). Together, the in vivo xenograft studies indicated that ALK inhibitor WHI-P154 could be a potential therapeutic agent for treatment of ALK mutation-induced lung tumorigenesis.

In lung metastasis model, H694R or E1384K mutation co-transfected with GFP-expressed construct in H1299 transfectants were used for detection of systemic metastasis after tail vein injection. Our results demonstrated that both H694R and E1384K mutations showed strong metastatic potential as compared with wild type and mock controls (FIG. 4D). Treatment with WHI-P154 to the mutation-bearing mice significantly suppresses lung metastasis (FIG. 4D). More importantly, mice with lung metastasis from both H694R and E1384K mutations showed poor survival rate (FIG. 4E). The potent metastatic potential of E1384K mutation resulted in poor mice survival is statistically significant as compared with wild type and WHI-P154 treated mutation-bearing mice (P=0.0246). In contrast, WHI-P154-treated mice with H694R and E1384K mutations not only suppress lung metastasis but also survive as long as control mice (FIG. 4E). We concluded that ALK mutations could constitutively activate ALK downstream oncogenic signalings resulted in tumorigenesis of lung adenocarcinoma. Targeting ALK mutations by ALK inhibitor WHI-P154 not only suppress tumorigenesis and metastasis but also prolong survival of mutation-bearing mice.

### Discussion

Our results demonstrated that ALK somatic alterations including aberrant increase of Y1604 phosphorylation and point mutations play pivotal roles in tumorigenic progression of lung cancers. Long-term overexpression of Y1604 phosphorylation of ALK could moderately activate downstream tumorigenic signaling pathways to mediate tumorigenic effects in lung cancer. Point mutations of ALK resulted in constitutive kinase activity to strongly enhance downstream AKT, STAT3 and ERK signaling pathways for driving tumor progression. Our results suggested that ALK kinase could be activated through heterogeneous pathways to increase phosphorylation of Y1604 and attributed in the early stage and throughout the progression of lung tumorigenesis. Nevertheless, aberrant increase of Y1604 phosphorylation and point mutations of ALK could potentially serve as diagnosis biomarker and therapeutic target for lung cancer, respectively.

Notably, in comparison with previous Western blot and IHC analysis that endogenous ALK protein expression was barely detected in lung tissues²⁴⁻²⁵, we also failed to detect endogenous ALK in Western blot analysis in lung cancer cell lines but were able to detect total ALK expression using IHC analysis in 61.59% (162/263) of lung cancer samples in tissue microarrays. Moreover, our detection of phospho-Y1604 ALK in lung cancer cell lines and tissues suggested that activation of ALK kinase through phosphorylation of Y1604 indeed occurred in lung cancers. We reasoned that the increased sensitivity to detect endogenous ALK and phospho-Y1604 ALK in lung cancers are possibly due to the aberrant up-regulation of phospho-Y1604 ALK, the use of antibodies from different suppliers and the applied of polymer-based signal amplification system instead of conventional method for IHC analysis. In addition to point mutations of ALK mediated constitutive activation of tyrosine kinase, future exploring of heterogeneous mechanisms to increase expression of tumorigenic phospho-Y1604 ALK and other cross-talked tumorigenic pathways should be warrant for revealing novel therapeutic targets in lung cancer.

Increased sensitivity of H694R and E1384K mutant versus wild type ALK to specific shRNA knockdown and ALK inhibitor WHI-P154 in various functional assays further suggested that the acquired somatic mutations with constitutive ALK kinase activity could be addictive for lung cancer cells to gain advantage of malignancy and are therefore served as a suitable target for the treatment of lung adenocarcinomas (FIG. 4 and Figure S6). We also examined the potential overall benefit of treating H694R and E1384K mutations with and without treatment of ALK inhibitor WHI-P154 using systemic metastasis in nude mice as a model by measuring the metastatic potential and survival. Although molecular mechanisms of suppressing cancer metastasis by WHI-P154 remained unclear, prolonged survival of H694R and E1384K mutation-bearing mice after treatment of WHI-P154 clearly suggested the therapeutic benefits of ALK inhibitor in lung cancer.

To further delineate the importance of ALK somatic alterations in diagnostic improvement and in its therapeutic benefits to lung cancer patients, several tasks need to be conducted in immediate future. First of all, phosphorylation activated ALK and other ALK mutations should be closely examined in larger cohorts and across different ethnic populations with various exposures of risk factors for potential disparities. Secondly, efforts should be given to study the etiological mechanisms of aberrant ALK phosphorylations and mutations that altered protein structures, enhanced their tyrosine kinase activity and mediated downstream oncogenic signaling pathways. The results will improve not only our understanding of the heterogeneous nature ALK signalings in tumor formation but also clinical management of ALK-mutated lung cancer patients. Finally, since ALK inhibitor WHI-P154 inhibited tumor progression and prolonged survival in mouse lung cancer models, our results clearly illustrate the great potential of ALK inhibitor as therapeutic tools for treating lung cancer and should facilitate development of new ALK inhibitors for personalized lung cancer treatment. Furthermore, lung cancer patients who harbored mutations of two other genes (ex. EGFR and KRAS) could potentially develop new therapeutic modalities to combine two clinically approved drugs for improving current lung cancer therapy.

### Materials and Methods

### Antibodies and Reagents

For Western blotting analysis, the membranes were probed with specific antibodies against HA (Covance), phospho-tyrosine (4G10; Upstate Biotechnology), STAT3 (sc-482; Santa Cruz) and α-tubulin (DM1A; NeoMarkers). The phospho-ALK (Tyr1604), phospho-Akt (Ser427), phospho-STAT3 (Tyr705), phospho-ERK (Thr202/Tyr204), Akt and ERK antibodies were purchased from Cell Signaling. For IHC staining assay, the tissue sections were stained with specific antibodies against phospho-ALK (pY1604; Epitomics), ALK (4204-1; Epitomics), phospho-STAT3 (Tyr705, D3A7; Cell Signaling) and phospho-Akt (Ser473, 736E11; Cell Signaling). The ALK inhibitor WHI-P154 was purchased from Calbiochem. The exon and intron boundaries of ALK were based on annotations in Ensembl database and their primers were provided in Table 1.

### ALK Constructs and Cell Transfection

The full-length cDNA of ALK-wild type was inserted into pCDNA3.0 vector by restriction sites Hind III and Not I. All ALK mutations were generated from the template of pCDNA3.0-ALK-wild type full-length construct using site-directed mutagenesis. H1299 and NIH3T3 cells were maintained in Dulbecco's Modified Eagle Medium (DMEM) medium supplemented with 10% Fetal Bovine Serum (FBS) and streptomycin. The H1299 and NIH3T3 cells were transfected with the expression constructs by Lipofectamine 2000 (Invitrogen) and further selected by G418 to establish mixtures of ALK-expressed transfectants.

### Western Blotting and Co-Immunoprecipitation Analysis

Cells were lysed in RIPA buffer (200 mM Tris, 150 mM NaCl, 1 mM EDTA, 1% NP40, 1 mM PMSF, 1 mM DTT) with addition of 1× phosphatase inhibitor cocktail set II from Calbiochem and 1× cocktails of protease inhibitor from Roche. The protein concentration was measured by BCA protein assay kit (Pierce Chemical). The equal amounts of cell lysates were subjected to SDS-PAGE, transferred to NC membranes, and then probed with specific antibody for protein detection. For co-immunoprecipitation assay, the equal amounts of cell lysates were firstly incubated with anti-HA antibody for one hour and subsequently reacted with protein A/G for overnight. The immunoprecipitated-beads were washed and subjected to Western blotting analysis using specific antibody.

### Immunohistochemistry

Human lung cancer tissue arrays containing 37 normal lung and 263 lung cancer sections from Pantomics (Luc961, Luc962, Luc1501, Luc1502 and Luc1503) were deparaffinized and the frozen tumor sections from the xenograft nude mice were fixed in 3% formaldehyde solution. All sections were treated in 3% H₂O₂ buffer for 30 minutes to inactivate the endogenous peroxidase activities, and then treated with 0.01M sodium citrate buffer for antigen retrieval. After blocking with 10% normal goat serum, these sections were reacted with specific antibodies at 4° C. for overnight. Subsequently, these sections were incubated with Super Picture HRP polymer conjugate secondary antibody (Zymed Laboratories), and then stained with DAB chromagen (Zymed Laboratories). These sections were also stained with Hematoxylin (DAKO).

### Cell Proliferation Assay

For analysis of cell growth, a total of 1×10³ cells/well was seeded in 96-well plate. After different culturing time, 10 μl/well WST-1 reagent (Roche Diagnostics) was added for reaction at 37° C. for 50 minutes. The absorbance at 450 nm was measured.

### Anchorage-Independent Growth Assay

A total of 2×10⁴ H1299 or NIH3T3 transfectants were mixed in a final 0.3% agarose and plated onto the 60-mm plate containing 0.5% agarose. These cells were further cultured in 5% CO₂ incubator at 37° C. for one month. The plates were dehydrated at room temperature and then stained with 0.3% crystal violet for visualizing colonies. The colony numbers were counted by using Image-pro plus analysis program.

### Boyden Chamber Assay

The cell migration capability was examined by Boyden chamber assay. Briefly, a total of 2×10⁴ H1299 or NIH3T3 transfectants were inoculated into the cell migration insert (BD Biosciences) containing 350 μl DMEM medium alone, and the insert was placed into the well containing 750 μl complete DMEM medium in 24-well plate. After 24 hours incubation, the migrated cells were fixed with 100% methanol and stained by Giemsa's solution (Merck). The numbers of migrated cells were counted by using Image-pro plus analysis program.

### In Vivo Xenograft Tumor Formation Assay

Animal protocol was approved by the Institutional Animal Safety Committee. A total of 1×10⁶ ALK-wild type or mutants expressed H1299 transfectants were mixed with matrigel (BD) and then subcutaneously injected into the right flank of four-week-old BALB/c NU mice. For WHI-P154 treatments, the nude mice were randomly divided into two groups when the mean tumor volume reached 20-50 mm³. DMSO or WHI-P154 (1 mg/kg/day) was intravenously injected daily. The tumor volumes were measured weekly and calculated according to the formula: volume=length×width²×0.52.

### In Vivo Metastasis Assay

In order to examine the in vivo metastatic capability, ALK wild type or mutations expressed H1299 transfectants were infected with GFP-lentivirus to generate the GFP fluorescence-labeled cells. A total of 2×10⁶ cells were injected into nude mice through tail vein for measuring lung metastasis. For investigation of the tumor suppressive effects using ALK inhibitor, the nude mice were intravenously injected daily with WHI-P154 (1 mg/kg/day) after cell inoculation for 14 days. The survival rate was recorded daily and scarified the rest mice after 105 days. The pulmonary metastasis of GFP-labeled H1299 transfectants were visualized by fluorescence stereomicroscope.

### Statistical Analysis

All data were presented as the standard error of the mean (mean±s.e.m). For the comparison of different group, Student's t tests were used to determine the statistical significance. For IHC expression correlation between phosphorylated-Y1604 and total ALK, Pearson's correlation coefficient was calculated in SAS. For survival analysis of mice, we applied a multiple-comparison adjustment to the P-values for the paired comparison between the wild type with each of the other groups was performed in SAS.

### Supplementary Materials and Methods

### In Vitro Kinase Assay

The in vitro kinase activity of ALK H1299 transfectants was measured by protein tyrosine kinase assay kit (Sigma) according to manufacturer's instructions. In brief, cells were lysed with lysis buffer. After protein quantification by BCA assay, equal amount of cell lysates was added into the wells coated with poly-Glu-Tyr substrate. After 30 min incubation, the plates were reacted with a peroxidase-conjugated anti-phosphotyrosine antibody. After incubation with OPD peroxidase substrate, the wells were read in a multiwell plate ELISA reader set at 492 nm absorbance.

### ShRNA and Lentivirus Infection

The shRNA for silencing ALK gene obtained from National RNAi Core Facility of Taiwan was constructed in pLKO.1 lentiviral expressing vector with a targeted sequence of CTGGTCATAGCTCCTTGGAAT. The shRNA against luciferase was used as control. For lentivirus production, 293T cells were co-transfected with lentiviral expressing vector, packaging plasmids pMD.G and pCMVΔR8.91. After four days transfection, the virus-containing media were collected and filtered. The 111299 ALK transfectants were infected with virus-containing media in the presence of polybrene. The shRNA knockdown efficiency was further evaluated by Western blotting analysis.

### DESCRIPTIONS OF THE DRAWINGS AND TABLES

### Figure Legends

FIG. 1: Tumorigenic effects of six ALK mutations. (A) ALK mutations were annotated along the schematic diagram of ALK protein structure containing two meprin/A5-protein/PTPmu (MAM) domains, one LDL Receptor class A (LDLA) domain, one Glycine-rich region, a transmembrane domain (TM) and a cytoplasmic tyrosine kinase (TK) domain. Identified ALK mutations were expressed in H1299 cells for comparing the tumorigenicity with mock transfection control in xenograft tumor formation assay. * and ** indicate statistical significance P<0.05 and P<0.01, respectively. (B) Mutation-bearing tissue sections over-expressed phosphor-Y1604 ALK using immunohistochemical analysis. Scale bars represent 50 μm.

FIG. 2: ALK somatic mutations and phosphorylation activate oncogenic signalings in lung cancer. (A) Immunoprecipitation (IP) and Western blot analysis of ALK wild type and mutations in H1299 and NIH3T3 transfectants. IP was performed with anti-HA antibody and subjected to Western blot analysis for detection of phospho-Y1604 and overall tyrosine phosphorylation (4G10) of ALK. Western blot analysis of ALK downstream signaling mediators including the phosphorylated proteins of STAT3, Akt and ERK were included with ALK expression control detected by anti-HA antibody, loading control of tubulin and total protein controls of STAT3, Akt, ERK. (B) Increased ALK Y1604 phosphorylation was detected in 11 lung cancer cell lines using Western blot analysis. (C) Distribution of phospho-Y1604 and total ALK expression intensities in normal and lung cancer specimens by IHC staining with scoring intensities from 0 to 4. Tissue arrays contained most subtypes of lung cancer (total cases-263) including small cell lung cancers (SCLC), adenocarcinomas (Ad), squamous cell carcinomas (SQ) and other subtypes. The red lines display the intensity by mean±s.e.m. ** stands for statistical significance of P<0.001.

FIG. 3. Transforming activity of ALK mutations H694R and E1384K. Oncogenic characteristics of wild type and mutant ALKs in H1299 and NIH3T3 transfectants were presented including cell proliferation (A), AIG (B, top panel) and cell migration capacity (B, bottom panel). (C) The tumor volume of xenografted ALK H1299 transfectants in nude mice was determined after 5 weeks subcutaneous inoculation displayed in photography (top panel) and in tumor growing curves (bottom panel). (D) Detection of Activated ALK downstream oncogenic signalings using IHC analysis of phosphorylated forms of ALK, STAT3 and Akt in xenograft tumor sections from wild type, H694R and E1384K ALK H1299 transfectants. Scale bars represent 50 μm. Data were shown as mean±s.e.m. * and ** indicate statistical significance P<0.01 and P<0.001, respectively.

FIG. 4. Therapeutic effects of ALK inhibitor WHI-P154 to H694R and E1384K mutations-mediated tumorigenesis. (A) The comparison of WHI-P154 inhibitory effects on H1299 transfectants of wild type, H694R and E1384K mutations including inhibitory effects of cell growth in dose-dependent manner by WST-1 assay (top panel), AIG displayed in colony numbers (middle panel) and cell migration in migrated cell numbers (bottom panel). (B) Dose-dependent inhibition of phosphorylated-Y1604 ALK expression by WHI-P154 treatments on H1299 transfectants of wild type, H694R and E1384K mutations were demonstrated by Western blotting analysis (top panel) and their quantitative intensities (bottom panel). (C) The tumor suppressive effect of WHI-P154 (1 mg/kg) on H694R or E1384K-induced tumorigenicity in xenograft tumor nude mice model was shown in photography (top panel) and in tumor growing curves (bottom panel). The effects of systemic lung metastasis models after 62 days tail vein injections of GFP-bearing H1299 transfectants of wild type, H694R and E1384K mutations with or without WHI-P154 treatments were visualized in macroscopic and fluorescent views of lung images (D) and in survival analysis (E).

FIG. 5. Electropherograms of six ALK somatic mutations. Snapshots of mutations were presented after alignment of sequencing data from the paired tumor and tumor-adjacent normal tissues of lung adenocarcinoma with majority of forward and reversed sequencing validation.

FIG. 6. In vitro kinase activity of ALK mutations H694R and E1384K was measured by ELISA reactions at 492 nm. Data were shown as mean±s.e.m. ** indicated the statistical significance of P<0.001.

FIG. 7. The protein stability and subcellular localization of mutant H694R and E1384K ALK. (A) The expression of HA-tagged wild type, H694R and E1384K ALK proteins in H1299 transfectants was examined in the presence of cycloheximide and Western blot analysis using antibody against HA (left panel). Addition of MG132 to stop protein degradation is used as positive control. The remaining protein quantification was shown in right panel and the half-life of ALK protein was labeled with dash line. (B) ALK transfectants were fixed in 4% formaldehyde/PBS and permeabilized in 0.5% Triton X-100/PBS. The TRITC-HA-labelled ALK proteins were visualized by confocal laser scanning microscopy. The nuclei were counterstained with Hoechst dye.

FIG. 8. Representative images of IHC scoring intensity of phosphorylated-Y1604 (A) and total (B) ALK of lung cancer sections in tissue arrays ranked from 0 to 4.

FIG. 9. The inhibitory effect of WHI-P154 on phosphorylated Y1604-ALK expression in xenograft tumor sections. DMSO treatment is used as control.

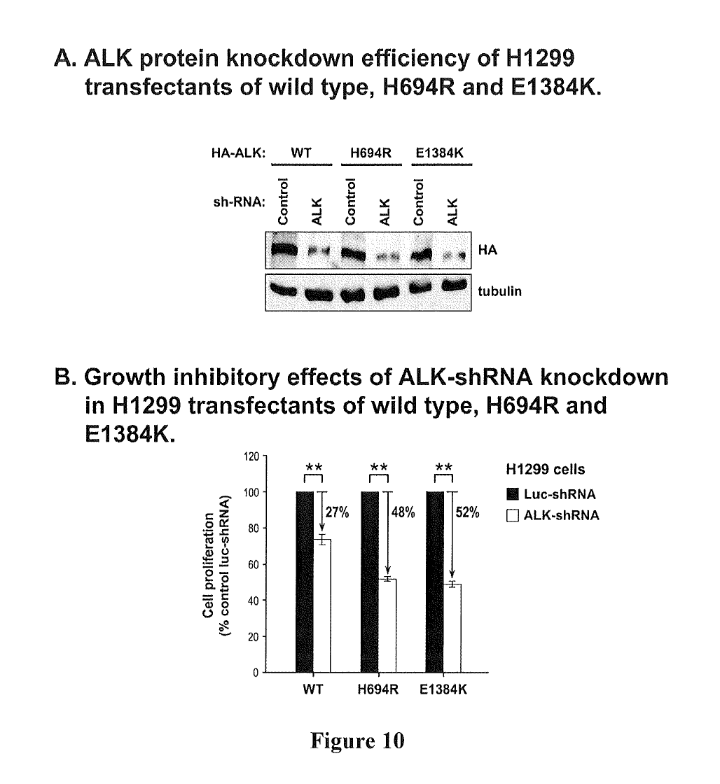

FIG. 10. The shRNA knockdown effect of ALK protein expression and cell proliferation in H1299 transfectants of wild type, H694R and E1384K. (A) ALK protein knockdown efficiency of H1299 transfectants by Western blot analysis. The tubulin was used as protein loading control. (B) Growth inhibitory effects of ALK-shRNA knockdown in H1299 transfectants of wild type, H694R and E1384K as compared with luciferase-shRNA control after 4 days lentivirus infection.

### TABLES

TABLE 1
List and sequences of sequencing primers of exons of ALK.

| SEQ. I.D. | PRIM ER NAME | SEQUENCE | SEQ. I.D. | PRIMER NAME | SEQUENCE |
|---|---|---|---|---|---|
| 001 | ALK01-1F | TCTGGAGATCAGGTGGAAGG | 056 | AL K15nest-R | ATCCCAGGTGCCAGTGACTA |
| 002 | ALK01-1R | TGAACAGCTCGCTGAGAT | 057 | ALK16ex-F | GCCAGCATGGCTAATTGAAC |
| 003 | ALK01-2F | CCTCGCTCTTCCGTGTCTAC | 058 | ALK16ex-R | GGACTAAGCAGGGAGGGAGT |
| 004 | ALK01-2R | AACACTAAATCCCGGCACAC | 059 | ALK16nest-F | TCACGCAGGTTCCCTATCTC |
| 005 | ALKO2ex-F | TCAGGGTCCTGAGGTCAACT | 060 | ALK16nest-R | GGAGGGAGTGACACCTTGAA |
| 006 | ALKO2ex-R | ATAGGGAGCTGAGGGAATGC | 061 | ALK17ex-F | CCCAGTGACCCCCTAACTTT |
| 007 | ALKO2nest-F | CTCATCTTCCCGATGGATGT | 062 | ALK17ex-R | TTAGCTTGGTGGGAGGACTG |
| 008 | ALKO2nest-R | GCCCTGGAGCACTATGAATC | 063 | ALK17nest-F | CCCCCATGTTAAACTCTGCT |
| 009 | ALKO3ex-F | TGAAGGCCAACCTCCTAGTG | 064 | ALK17nest-R | GGGAGGACTGACCTAAGCAA |
| 010 | ALKO3ex-R | CCAAGAAGCCATGGAAAGTC | 065 | ALK18ex-F | TCCAGTGGCTATGGGACCTA |
| 011 | ALKO3nest-F | AAGTCACGTTTGGGAAATGG | 066 | ALK18ex-R | CATGACCCACCTTTCACACA |
| 012 | AL KO3nest-R | GACATCACCAGCAGCCTCTC | 067 | ALK18nest-F | CTAGGAACAGGGACCACCAG |
| 013 | AL KO4ex-F | GTCCACCTGCATCAGGCTAT | 068 | ALK18nest-R | AAAACCGAATCCAGGGTGTT |
| 014 | ALKO4ex-R | GAAGTCAGAGGGACCCACAA | 069 | ALK19ex-F | CTCATGGTCCCCTGAAAAGA |
| 015 | ALKO4nest-F | TGCAGATTCGTTTTCCCTGT | 070 | ALK19ex-R | TCACCATCGTGATGGACACT |
| 016 | ALKO4nest-R | CAGAGGGACCCACAAATCAG | 071 | ALK19nest-F | AAGGAGACCTGGTGTGGTTG |
| 017 | ALKO5ex-F | AAAAGGAATGCCAGTGGTGAG | 072 | ALK19nest-R | TCGTGATGGACACTGAAGGA |
| 018 | ALKO5ex-R | CCAAACATGGTTGCAGGTTA | 073 | ALK20ex-F | TCAGAGCTCAGGGGAGGATA |
| 019 | ALKO5nest-F | AGACTGGGTTCGTGAATTGG | 074 | ALK20ex-R | GAGTCTGCGGTGCTGTGATA |
| 020 | AL KO5nest-R | TGGTTGCAGGTTATTGACACA | 075 | ALK20 nest-F | GGAAGTGGCCTGTGTAGTGC |
| 021 | ALKO6ex-F | AGGGAACATGGACCACTCTGCTG | 076 | ALK2Onest-R | ACATTCAGCCCCTACACTGC |
| 022 | AL KO6ex-R | TGGGCATAGAGGACTTCCAATGTCA | 077 | ALK22_21ex-F | CCCAGCTGCCTCATTATTGT |
| 023 | AL KO6nest-F | ACCACTCTGCTGCTCACCTT | 078 | ALK22_21ex-R | PACCATCAAGGGITGITCCA |
| 024 | AL KO6nest-R | GCTGGGCTTTATCCTGCACAT | 079 | ALK22_2lnest-F | GCCTCATTATTGTGGCCTGT |
| 025 | AL KO7ex-F | TTGGGGGCTTCTCTTCATTA | 080 | ALK22_21nest-R | GGACAACACGATTTCCCTTG |
| 026 | ALKO7ex-R | GAAAGCCCAAGGTGTGAAGA | 081 | ALK23ex-F | ACCTGCTCACCAGCAAGATT |
| 027 | ALKO7nest-F | GATGGTGCCTCTCTGCTCTC | 082 | ALK23ex-R | TCCATTCTCTTCCAGCCAGT |
| 028 | ALKO7nest-R | GTGAAGTCCAGCCCTGAGTC | 083 | ALK23nest-F | CTGCTGCCCATGTTTACAGA |
| 029 | ALKO8ex-F | AGGTGGGCTTTCTTCCAT | 084 | ALK23nest-R | CTTCCATCCTTGCTCCTGTC |
| 030 | ALKO8ex-R | AGAGGTGGCCCTCTTGTTCT | 085 | ALK24ex-F | CATTTCCCCTAATCCTTTTCCA |
| 031 | ALKO8nest-F | GTTCCTGATAATGGCGTGCT | 086 | ALK24ex-R | GTGATCCCAGATTTAGGCCTTC |
| 032 | ALKO8nest-R | GGITCCGGAAGTGACAAGAG | 087 | ALK24nest-F | GGAAATATAGGGAAGGGAAGGN |
| 033 | ALKO9ex-F | TCTTGGTGAGACAGGTGGTG | 088 | ALK24nest-R | TTGACAGGGTACCAGGAGATGA |
| 034 | ALKO9ex-R | GAGAAGGGTATTGGGGGAGA | 089 | ALK25ex-F | GCCTCTCGTGGTTTGTTTTGTC |
| 035 | AL KO9nest-F | AAGGGGACCITTCAGCAATC | 090 | ALK25ex-R | CCCAGGGTAGGGTCCAATAATC |
| 036 | AL KO9nest-R | ATTGGGGGAGATGCATAGAG | 091 | ALK25nest-F | CTGAACCGCCAAGGACTCAT |
| 037 | ALK11_10ex-F | GGGATTAGCGAGCCTTTTTC | 092 | ALK25nest-R | TTTTCCCTCCCTACTAACACACG |
| 038 | AL K11_10ex-R | ACAGCTCCCCACCCTAAAGA | 093 | ALK26ex-F | CCTGCTCTCCTCCTGAACC |
| 039 | ALK11_10nest-F | TTGCTCCTCACACACATTCC | 094 | ALK26ex-R | CAGGGATACCTGGAGGATGA |
| 040 | ALK11-10nest-R | CAGCACCAATCTTTCTICTGC | 095 | ALK26nest-F | AGCAGGGCAGATGCTTAATG |
| 041 | ALK12ex-F | CATCTTGGATGGAGGGTTTG | 096 | ALK26nest-R | CTGGAGGATGATGGCTGACT |
| 042 | ALK12ex-R | ATCTCCCCATCTCCAACCTT | 097 | ALK27ex-F | GAATGIGGGTGGGIGTGTCT |
| 043 | ALK12nest-F | GACTGCCTCTCCTCTTGTGC | 098 | ALK27ex-R | CAGTCACATTCGCATCTTGG |
| 044 | ALK12nest-R | ATGCACCCATAGGCAAGACT | 099 | ALK27 nest-F | GGTCCTTTGGAGTGCTGCTA |
| 045 | AL Kl3ex-F | GAAGTGGGGGAGAAGAATCC | 100 | ALK27 nest-R | CCATCTGCCCCTTCATCTTA |
| 046 | AL K13ex-R | AACTTCCAGGAGGAGGGTGT | 101 | ALK28ex-F | CCTTTACACCTGCGCACTCT |
| 047 | AL K13nest-F | AGAACCCTCAGACCACGATG | 102 | ALK28ex-R | AAATGGGCAAATGGAGACAC |
| 048 | ALK13nest-R | AGGAGGGTGTTGAGTGTTGG | 103 | ALK28nest-F | GTTCGCACCCTCAACGTATT |
| 049 | ALK14ex-F | TTCTGTCTGCTGCAAAGTGG | 104 | ALK28nest-R | GCAAATGGAGACACCAGTCA |
| 050 | ALK14ex-R | TCATGAGGCTCTGACATTGC | 105 | AL K29-1F | AAATCCTGGTTTCCTCATCTG |
| 051 | ALK14nest-F | GCCACAAACCTAACGTGCTT | 106 | ALK29-1R | GTGTGGCTCCTTCTTTGCTA |
| 052 | ALK14nest-R | ATCTGGCAGCACACACCATA | 107 | ALK29-2F | AAGGGGGACACGTGAATATG |
| 053 | ALK15ex-F | GAAGCACAGCTCGGTTTCTC | 108 | ALK29-2R | TIGGCACAAAACAAAACGTG |
| 054 | ALK15ex-R | AGCTCCAGGTCAGCAAGATG | | | |
| 055 | AL K15nest-F | TCTGAATGTCTCCCCTGGTC | | | |

TABLE 2
The correlation between phosphorylated-Y1064
and total ALK expression in 263 lung cancers
specimens in tissue arrays using IHC analysis

| IHC staining antibody | n | IHC density | P-value |
|---|---|---|---|
| p-ALK | 263 | 2.9684 ± 0.6852 | |
| ALK | 263 | 1.2274 ± 0.7707 | 0.4449 |

Data shows Mean ± SD
No correlation between p-ALK and ALK (Pearson's coefficient r = 0.04703)

TABLE 3
The IC50 concentrations of WHI-P154-induced cell growth inhibition
in H1299 transfectants of wild type, H694R and E1384K ALKs.

| | IC₅₀ (cell proliferation |
|---|---|
| ALK | inhibition by WHI-P154) |
| WT | 26.9375 |
| H694R | 11.7901 |
| E1384K | 9.4238 |

The foregoing description of the invention illustrates and describes embodiments of the present invention. It is to be understood that the invention is capable of use in various other combinations, modifications, and environments and is capable of changes or modifications within the scope of the inventive concept as expressed herein, commensurate with the above teachings and/or the skill or knowledge of the relevant art. The embodiments described above are further intended to explain best modes known of practicing the invention and to enable others skilled in the art to utilize the invention in such, or other, embodiments and with the various modifications required by the particular applications or uses of the invention. Accordingly, the description is not intended to limit the invention to the form or application disclosed herein.

### REFERENCES

1. WHO. World Health Organization. “Cancer”. http://www.who.int/mediacentre/factsheets/fs297/en/. Retrieved 2010 Mar. 3.
2. Ladanyi, M. & Pao, W. Lung adenocarcinoma: guiding EGFR-targeted therapy and beyond. Mod Pathol 21 Suppl 2, S 16-22 (2008).
3. Sun, S., Schiller, J. H., Spinola, M. & Minna, J. D. New molecularly targeted therapies for lung cancer. J Clin Invest 117, 2740-2750 (2007).
4. Herbst, R. S., Heymach, J. V. & Lippman, S. M. Lung cancer. N Engl J Med 359, 1367-1380 (2008).
5. Sun, S., Schiller, J. H. & Gazdar, A. F. Lung cancer in never smokers—a different disease. Nat Rev Cancer 7, 778-790 (2007).
6. Subramanian, J. & Govindan, R. Molecular genetics of lung cancer in people who have never smoked. Lancet Oncol 9, 676-682 (2008).
7. Jemal, A., et al. Cancer statistics, 2009. CA Cancer J Clin 59, 225-249 (2009).
8. Fischer, P., et al. A Ki-1 (CD30)-positive human cell line (Karpas 299) established from a high-grade non-Hodgkin's lymphoma, showing a 2;5 translocation and rearrangement of the T-cell receptor beta-chain gene. Blood 72, 234-240 (1988).
9. Morris, S. W., et al. Fusion of a kinase gene, ALK, to a nucleolar protein gene, NPM, in non-Hodgkin's lymphoma. Science 263, 1281-1284 (1994).
10. Chiarle, R., Voena, C., Ambrogio, C., Piva, R. & Inghirami, G. The anaplastic lymphoma kinase in the pathogenesis of cancer. Nat Rev Cancer 8, 11-23 (2008).
11. Soda, M., et al. Identification of the transforming EML4-ALK fusion gene in non-small-cell lung cancer. Nature 448, 561-566 (2007).
12. Soda, M., et al. A mouse model for EML4-ALK-positive lung cancer. Proc Natl Acad Sci USA 105, 19893-19897 (2008).
13. Horn, L. & Pao, W. EML4-ALK: honing in on a new target in non-small-cell lung cancer. J Clin Oncol 27, 4232-4235 (2009).
14. Mosse, Y. P., et al. Identification of ALK as a major familial neuroblastoma predisposition gene. Nature 455, 930-935 (2008).
15. Janoueix-Lerosey, I., et al. Somatic and germline activating mutations of the ALK kinase receptor in neuroblastoma. Nature 455, 967-970 (2008).
16. Chen, Y., et al. Oncogenic mutations of ALK kinase in neuroblastoma. Nature 455, 971-974 (2008).
17. George, R. E., et al. Activating mutations in ALK provide a therapeutic target in neuroblastoma. Nature 455, 975-978 (2008).
18. Palmer, R. H., Vernersson, E., Grabbe, C. & Hallberg, B. Anaplastic lymphoma kinase: signalling in development and disease. Biochem J 420, 345-361 (2009).
19. Balsara, B. R. & Testa, J. R. Chromosomal imbalances in human lung cancer. Oncogene 21, 6877-6883 (2002).
20. Tseng, R. C., et al. Genomewide loss of heterozygosity and its clinical associations in non small cell lung cancer. Int J Cancer 117, 241-247 (2005).
21. Weir, B. A., et al. Characterizing the cancer genome in lung adenocarcinoma. Nature 450, 893-898 (2007).
22. Bai, R. Y., Dieter, P., Peschel, C., Morris, S. W. & Duyster, J. Nucleophosmin-anaplastic lymphoma kinase of large-cell anaplastic lymphoma is a constitutively active tyrosine kinase that utilizes phospholipase C-gamma to mediate its mitogenicity. Mol Cell Biol 18, 6951-6961 (1998).
23. Amin, H. M., et al. Inhibition of JAK3 induces apoptosis and decreases anaplastic lymphoma kinase activity in anaplastic large cell lymphoma. Oncogene 22, 5399-5407 (2003).
24. Boland, J. M., et al. Anaplastic lymphoma kinase immunoreactivity correlates with ALK gene rearrangement and transcriptional up-regulation in non-small cell lung carcinomas. Hum Pathol 40, 1152-1158 (2009).
25. Martelli, M. P., et al. EML4-ALK rearrangement in non-small cell lung cancer and non-tumor lung tissues. Am J Pathol 174, 661-670 (2009).

What is claimed is:

1. A method of diagnosing lung cancer comprising steps of detecting ALK mutations from lung adenocarcinoma patients and detecting various tumorigenic activities through a panel of assays.

2. The method as in claim 1, wherein the ALK mutation is S413N.

3. The method as in claim 1, wherein the ALK mutation is V597A.

4. The method as in claim 1, wherein the ALK mutation is H694R.

5. The method as in claim 1, wherein the ALK mutation is G8811D.

6. The method as in claim 1, wherein the ALK mutation is Y1239H.

7. The method as in claim 1, wherein the ALK mutation is E1384K.

8. The method as in claim 1, wherein expression of phosphorylated-Y1604-ALK is detected.

9. The method as in claim 1, wherein increased expression of phosphorylated-Y1604-ALK is detected.

10. The method as in claim 8, wherein the expression of phosphorylated-Y1604-ALK is detected by an IHC assay.

11. The method as in claim 8, wherein the average phosphorylated-Y1604-ALK scoring intensity is 0.554±0.3340 for normal lung tissues while the average phosphorylated-Y1604-ALK scoring intensity is increased to 2.9684±0.6852 for lung cancer tissues.

12. The method as in claim 8, wherein sensitivity and specificity of phosphorylated-Y1604-ALK antibody for detecting lung cancer are 92.8% and 100%, respectively, when IHC intensity is >2.

13. The method as in claim 8, wherein sensitivity and specificity of phosphorylated-Y1604-ALK antibody for detecting lung cancer are 99.6% and 89.2%, respectively, when IHC intensity is >1.

14. The method as in claim 1, wherein a panel of assays on non-synonymous ALK mutations including ALK kinase activation, cell proliferation, migration, and anchorage-independent growth.

15. The method as in claim 11, wherein in vivo mouse xenograft tumorigenesis assay is included.

16. A method of treating lung cancer comprising of steps of detecting ALK mutations from lung adenocarcinoma patients and suppressing ALK mutations-mediated tumorigenesis by administering an ALK inhibitor.

17. A method as in claim 16, wherein the ALK inhibitor is selected from WHI-P154, TAE684 and Crizotinib.

18. A method of diagnosing lung cancer comprising steps of detecting ALK mutations from lung adenocarcinoma patients and detecting various tumorigenic activities through a panel of assays wherein
the detecting ALK mutations from lung adenocarcinoma patients is up-regulated expression of phosphorylated-Y1604-ALK, and
the expression of phosphorylated-Y1604-ALK is detected by an IHC assay.

19. The method as in claim 18, wherein the average phosphorylated-Y1604-ALK scoring intensity is 0.554±0.3340 for normal lung tissues while the average phosphorylated-Y1604-ALK scoring intensity is increased to 2.9684±0.6852 for lung cancer tissues.

20. The method as in claim 18, wherein sensitivity and specificity of phosphorylated-Y1604-ALK antibody for detecting lung cancer are 92.8% and 100%, respectively, when IHC intensity is >2.

21. The method as in claim 18, wherein sensitivity and specificity of phosphorylated-Y1604-ALK antibody for detecting lung cancer are 99.6% and 89.2%, respectively, when IHC intensity is >1.

22. The method as in claim 18, wherein a panel of assays on non-synonymous ALK mutations including ALK kinase activation, cell proliferation, migration, and anchorage-independent growth.

23. The method as in claim 19, wherein in vivo mouse xenograft tumorigenesis assay is included.

24. A method of treating lung cancer comprising of steps of detecting ALK mutations from lung adenocarcinoma patients and suppressing ALK mutations-mediated tumorigenesis by administering an ALK inhibitor wherein
the detecting ALK mutations from lung adenocarcinoma patients is up-regulated expression of phosphorylated-Y1604-ALK,
the expression of phosphorylated-Y1604-ALK is detected by an IHC assay, and
the ALK inhibitor is selected from WHI-P154, TAE684 and Crizotinib.

25. The method as in claim 24, wherein the average phosphorylated-Y1604-ALK scoring intensity is 0.554±0.3340 for normal lung tissues while the average phosphorylated-Y1604-ALK scoring intensity is increased to 2.9684±0.6852 for lung cancer tissues.

26. The method as in claim 24, wherein sensitivity and specificity of phosphorylated-Y1604-ALK antibody for detecting lung cancer are 92.8% and 100%, respectively, when IHC intensity is >2.

27. The method as in claim 24, wherein sensitivity and specificity of phosphorylated-Y1604-ALK antibody for detecting lung cancer are 99.6% and 89.2%, respectively, when IHC intensity is >1.

28. The method as in claim 24, wherein a panel of assays on non-synonymous ALK mutations including ALK kinase activation, cell proliferation, migration, and anchorage-independent growth.

29. The method as in claim 25, wherein in vivo mouse xenograft tumorigenesis assay is included.

30. A method as in claim 17, wherein the ALK inhibitor is WHI-P154.

31. A method of treating lung cancer as in claim 24, wherein the ALK inhibitor is WHI-P154.
